(19) United States
(12) Patent Application Publication
(10) Pub. No.: US 2003/0135154 A1
(43) Pub. Date: Jul. 17, 2003

(54) Catheter head comprising a flow sensor

(76) Inventors: Hanspeter Heiniger, Lotzwil (CH); Simone Geiser, Langenthal (CH); Michael Krieftewirth, Bern (CH); Gilbert Schiltges, Kirchberg (CH); Julian Yeandel, Grosshoechstetten (CH)

Correspondence Address: David E. Bruhn, DORSEY & WHITNEY LLP, Suite 1500, 50 South Sixth Street, Minneapolis, MN, 55402-1498, US

(21) Appl. No.: 10/325,472

(22) Filed: Dec. 19, 2002

Related U.S. Application Data
(63) Continuation of application No. PCT/CH01/00405, filed on Jun. 29, 2001.

(30) Foreign Application Priority Data
Jul. 20, 2000 (DE) 100 35 342.8

(51) Int. Cl. A61M 31/00

(52) U.S. Cl. 604/67

(57) ABSTRACT

The invention relates to a device for supplying liquid into a body, the device including a catheter head, a liquid supply to the catheter head and a means for measuring the flow of the liquid, wherein the means for measuring the flow is a flow sensor arranged directly on or in the catheter head.

### PRIORITY CLAIM

This application is a continuation of International Patent Application PCT/CH01/00405, filed on Jun. 29, 2001, which claims priority to German Patent Application DE 100 35 342.8, filed on Jul. 20, 2000, both of which are hereby incorporated by reference.

### BACKGROUND

The present invention relates to a device for supplying liquid into the body, comprising a catheter head, a liquid supply to the catheter head and a means for measuring the flow of the liquid.

In order to be able to exactly monitor the administering of active agents into the body, it is necessary to measure the flow of the active agents using a catheter system. Various systems for this purpose are already known. U.S. Pat. No. 5,764,539, for example, proposes measuring the flow by attaching a temperature-dependent sensor to the catheter hose behind the pump, whose readings are processed electronically.

Another such thermo-flow sensor, likewise attached to the hose guiding the liquid, is known for example from EP 0 405 148 A1. For intravenous infusion systems, U.S. Pat. No. 5,445,622 proposes a system for measuring flow in which a flow indicator, designed as a separate wrist device, is attached to an infusion hose. Lastly, a flow sensor for an infusion pump is known from U.S. Pat. No. 5,462,525 which is arranged on the pump itself, namely at the discharge channel of the pump.

The disadvantage of all the systems cited above is that they can in no way detect leaks arising directly at or in the vicinity of the injection point. If a leak or blockage arises directly at the injection point into the body, with the result that the active agent liquid leaves the catheter in a way other than that provided for, a flow sensor arranged in accordance with the prior art will not detect this defect and a corresponding counter measure cannot (automatically) be introduced.

### SUMMARY

Against this background, it is the object of the present invention to provide a device for supplying liquid into the body which overcomes the disadvantages of the prior art mentioned above. In particular, the intention is to provide reliable flow measurement which can ascertain any possible defect in the flow of active agent and therefore provides the possibility of detecting the flow very exactly and correctly.

The advantages of the present invention are based on the fact that the means for measuring the flow is a flow sensor arranged directly on or in the catheter head. Thus, in accordance with the invention, the flow sensor is arranged at a point of the flow channel lying directly at the interface to the body, i.e., directly on or in the catheter head. This ensures that any change in the flow, which could occur over the entire liquid supply, is also registered by the flow sensor; any leaks, blockages or the flow of air bubbles or free-flow can be detected by means of the device in accordance with the invention. The system in accordance with the invention therefore operates far more reliably than all previously known arrangements, in which it has not been possible to register leaks downstream of the flow sensor. This also immediately presents the possibility of automatically regulating the device in response to changes in the flow.

In one embodiment of the present invention, the flow sensor is arranged downstream of a catheter supply line of the catheter head and in particular between the catheter supply line and a connecting needle of the catheter head which leads to a catheter cannula. It is thus arranged at the point lying directly before the part of the catheter head at which, through exact manufacture, leaks can be ruled out, and directly behind the part of the supply line in which, due to the possibility of leaking adapter connections, leaks can relatively easily arise. A miniaturized flow sensor can be attached and fixed to the point cited, and largely redesigning the catheter head is not necessary.

In order to monitor the data captured by the flow sensor, it is advantageous if a data transfer means is arranged on the device itself in accordance with the invention. This data transfer means can be directly integrated with the flow sensor, and in accordance with an advantageous embodiment of the invention, connects the flow sensor to a regulating means in a pump for the liquid, by means of which the flow is regulated to a predetermined value by comparing desired and actual values. As already remarked above, the device in accordance with the invention is particularly suitable, due to the optimal positioning of the flow sensor and the measuring accuracy which can therefore be achieved, for being incorporated into a regulating system which automatically corrects deviations detected by the flow sensor. If, at a given moment, for example, there is too little flow or air bubbles are detected in the channel, then the flow output of the pump can immediately be set higher via the regulating means, which then ensures a correct flow rate again. The active agent can then be administered again as planned. In some embodiments, a signal output means is also integrated which, when there are more significant or non-correctable deviations in the flow, emits a signal which prompts the operator to check the liquid supply.

In one embodiment, the present invention comprises a device for supplying liquid into a person's body, comprising a catheter head, a liquid supply, e.g., a conduit through which the liquid may flow, coupled to said catheter head, a flow sensor associated with the catheter head, a processing unit and a pump, said flow sensor, processing unit and pump operably coupled in a feedback loop.

In one technical embodiment, the flow sensor can comprise an analogue/digital-converter which is integrated on a chip and digitally transfers the data captured.

Data transfer can be realized in various ways in accordance with the invention. In one embodiment, the data transfer means comprises cables which lead from the flow sensor in the catheter head and in or on the components along the liquid supplies to a processing unit, in particular the regulating means on the pump, wherein line junctions at components of the liquid supply to be connected to each other are preferably realized by contacts, in particular touching contacts.

The entire data transfer means is thus integrated into the liquid supply, such that no additional, separate, external cabling is required. The advantage is that the patient only has a single connection from the catheter head to the pump. In their technical embodiment, the cables are directly worked into the catheter in a passageway and the catheter hose is simultaneously extruded together with the cables. In the catheter head, the cables are separated from the hose and soldered or fixed to the flow sensor. In the connectors, i.e., in the individual components/adapters provided with contacts, the cables are fixed to the contacts.

A further advantage of this embodiment is that, by checking that the electric contact is good, it is always possible to simultaneously monitor whether the individual components and adapters to be connected to each other have been correctly connected to each other.

In accordance with another embodiment of the present invention, the data transfer means comprises cables which lead separately from the liquid supply to a processing unit, in particular the regulating means on the pump. In this solution comprising separate cables, the liquid supply is simply designed conventionally, and it is possible to connect the cables to the catheter head and fix them there by means of a plug-on adapter.

In accordance with another embodiment of the data transfer means, the latter comprises a transmitter which wirelessly transfers the data captured by the flow sensor to a receiver on a processing unit, in particular the regulating means on the pump. The transmitter can be connectable and fixable to the catheter head by means of a plug-on adapter. Such wireless data transfer has, on the one hand, the advantage that the liquid supply lines do not have to be provided with specially integrated cables and contact connectors, and on the other hand again ensures the possibility of providing only a single line between the pump and the patient.

### BRIEF DESCRIPTION OF THE DRAWINGS

FIG. 1 depicts a catheter connector in accordance with the present invention, the components of the liquid supply being shown individually;

FIG. 2 depicts a catheter connector in accordance with the present invention with the components of the liquid supply assembled;

FIG. 3 depicts the arrangement set forth in FIG. 2, in a side view; and

FIG. 4 is a block diagram schematically representing the design of a wireless data transfer between the flow sensor and the pump.

### DETAILED DESCRIPTION

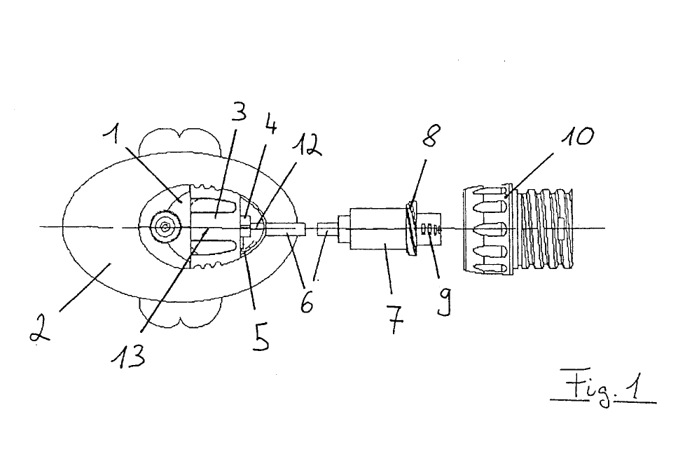

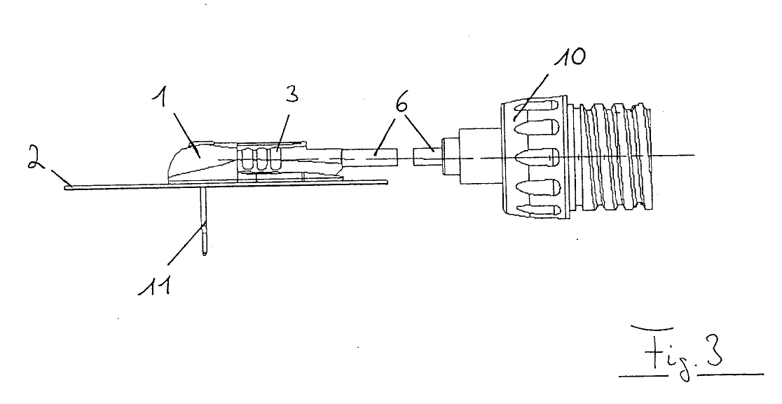

The individual components of a catheter connector in accordance with the invention shall now be described by way of FIGS. 1 to 3. The catheter head itself carries the reference numeral 1 and is fixed to the skin of a patient by means of a fixing fabric (plaster), such that the cannula 11 (see FIG. 3) penetrates to the point in the patient's body where the active agent is to be delivered.

A connecting needle 13 ensuring the supply of active agent into the cannula 11 runs in the catheter head, said needle lying beneath the removable connecting plug 3 onto which an adapter (not shown) can be plugged for data transfer.

The flow sensor 4, for example, a miniaturized flow meter for the smallest mass flows, is situated between the inlet of the connecting needle 13 and the catheter supply 12. For data output, the cables 5 proceed from the flow sensor 4, in some embodiments, preferably three cables for the data line and power supply.

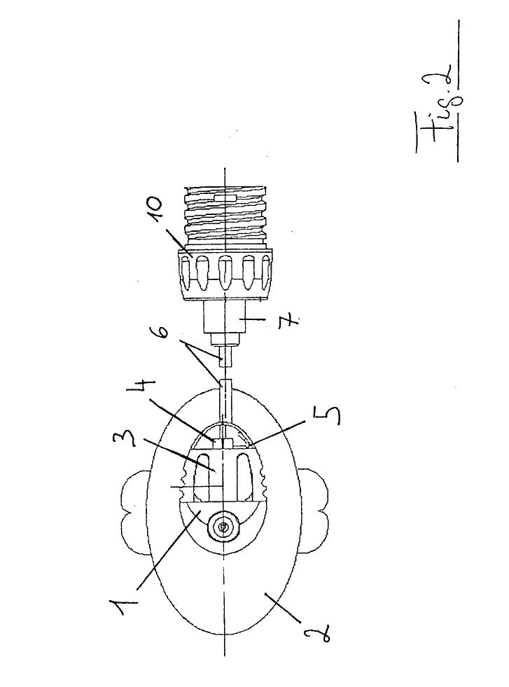

The embodiment shown is one which comprises a data line integrated into the liquid supply. Although not specifically shown in FIGS. 1 and 2, the data cables 5 run through the catheter hose 6 into the adapter 7 which is provided with a thread 8 and a Luer connector (not shown) arranged to the right of this. The data line terminates at the Luer connector in three contacts 9 which make contact with counter contacts (not visible) in the pump adapter 10 when the components 7 and 10 are connected. The data line is then continued via further integrated cables up to the pump (also not shown).

The active agent fluid thus flows via the components 10, 7, 6 and 12 into the flow sensor 4, and then via the connecting needle 13 into the cannula 11 where it is administered in the body. The flow sensor 4 reports the flow rate back via its data line, such that adjustments can be made in the event of irregularities in the flow rate. In the embodiment shown, because the data line is integrated into the liquid line, no separate and possibly disruptive data line is necessary.

FIG. 2 shows the arrangement in accordance with FIG. 1 in a state in which the adapters 7 and 10 have already been assembled, and in FIG. 3 this state may be seen in the side view.

At this point, reference should again be made to the connecting plug 3 such as is shown in FIGS. 1 to 3. In principle, the possibility exists of not guiding the cables 5 through the supply line but connecting them to the connecting plug 3 and leading off a separate data line cable, for data transfer, via this connection. In this case, it is not necessary to manufacture the liquid supply line with relatively expensive integrated cables and connectors, rather a conventional liquid supply line can be used.

The flow sensor is constructed in accordance with a so-called packaging principle. Amplifiers and analogue/digital converters are integrated on the chip, such that no other electronic components are necessary except the chip which includes the actual sensor. The data from the sensor are transferred digitally.

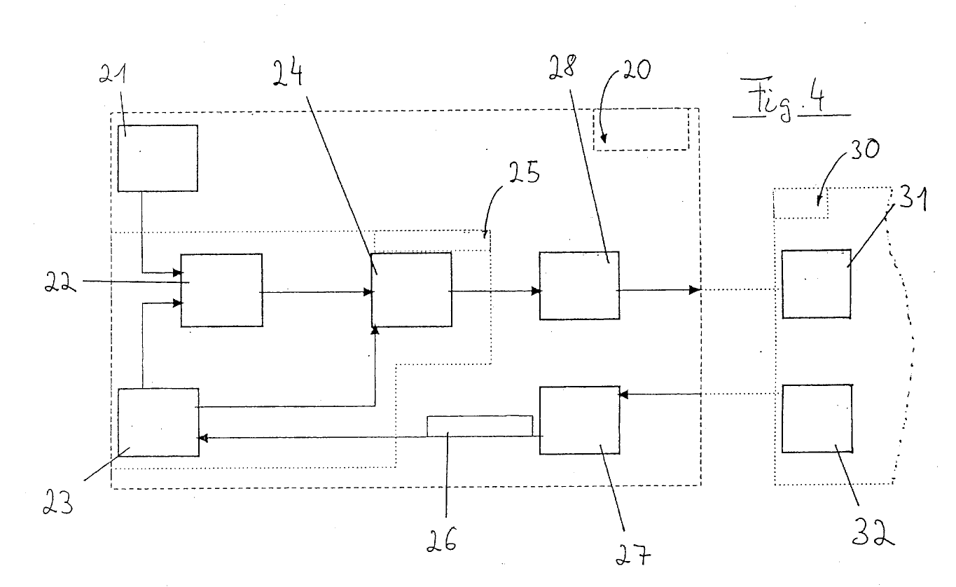

In accordance with another embodiment of the invention, a transmitter for wireless data transfer can also be attached to the connecting plug 3 shown in FIGS. 1 to 3, said transmitter then receiving the data from the flow sensor 4 via the cables 5 and wirelessly relaying them, i.e. for example, transmitting them to the pump. The transmitter is equipped with a corresponding electronic system (see FIG. 4) which allows the data to be transmitted encoded or only assigned to the respective pump. The transmitter can be equipped with a replaceable battery as its energy source and can be attached to the catheter head and detached again.

FIG. 4, already mentioned, shows a block diagram for the transmitter electronic system (transmitter) 20 and the pump input and output 30. The transmitting electronic system is integrated with the flow sensor 4 as described above. The part of the pump 30 shown on the right in FIG. 4 comprises a digital transmitter 32 and a digital receiver 31, for the digital signals transmitted wirelessly from the transmitter 20, respectively. In the transmitter 20, the sensor operation amplifier 21 is shown, which amplifies the signals received from the sensor 4 and relays them to the A/D converter 22. The A/D converter 22 is a component of a micro controller 25 which also includes the timing signal generator 23 and the data encoder 24. From the A/D converter, the data from the sensor 4—amplified in the operation amplifier 21—are delivered to the digital transmitter 28 of the transmitter 20 which then transmits them on to the digital receiver 31 of the pump 30.

In an electronic system (not shown) of the pump 30, the flow data is revised by means of comparing desired and actual values and, if a correction signal is necessary, this is delivered to the digital receiver 27 of the transmitter 20 via the digital transmitter 32. These correction data 26 are then relayed to the timing signal generator 23 which, via outputs to the A/D converter and to the encoder 24—directly or indirectly—transmits the new actual state to the pump via the digital transmitter 28. If the new actual state is within the prescribed range, taking into account the correction performed, i.e. a change in the liquid delivery of the pump, the medicine is administered again as planned. If this is not the case, a signal can be output which prompts the operator to manually check the entire catheter system.

With respect to the diagram in FIG. 4, the following may also be noted: the local energy supply for the flow sensor 4/transmitter 20 (to operate the sensor, it is necessary to generate a high voltage) is integrated as a part of the sensor operation amplifier module 21. Therefore, the battery voltage (in the range 1.2 to 1.5 V) can be used as a single, global energy supply. The operation amplifier 21 emits signals within the standard range of −10 to +10 V. The timing signal generator 23 can generate suitable signals to control a sampling of the analogue signals by the AID converter 22. Furthermore, the timing signal generator 23 can generate suitable signals to regulate the combination of sampled device identification data/flow data before digital transmission. A suitable, series interface exists between the micro controller 25 and the digital receiver 31 as well as the digital transmitter 32, in order to allow data processing. Furthermore, there exists economical encoding. This would consist for example of a combination of metal contacts printed onto a circuit board, and a conductive coating dye. This code is part of the transferred data generated by the micro controller 25.

A suitable program can be developed for operating the micro controller, said program taking care of any necessary coordination of data composition, communication and power supply management.

By arranging or associating the flow sensor directly on or with the catheter head, the invention allows the flow rate to be measured intensely, accurately and error-free, in particular relating to leaks and/or blockages in error-prone components such as connector adapters in the liquid supply before the catheter head.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for supplying liquid into a body, comprising a catheter head, a liquid supply to said catheter head and a means for measuring the flow of the liquid, wherein said means for measuring the flow is a flow sensor arranged directly on or in the catheter head.

2. The device as set forth in claim 1, wherein said flow sensor is arranged downstream of a catheter supply line of the catheter head.

3. The device as set forth in the preceding claim, wherein said flow sensor is arranged between the catheter supply line and a connecting needle of the catheter head which leads to a catheter cannula.

4. The device as set forth in claim 1, further comprising a data transfer means for data captured by the flow sensor.

5. The device as set forth in claim 4, wherein said data transfer means connects the flow sensor to a regulating means in a pump for the liquid, by means of which the flow is regulated to a predetermined value by comparing desired and actual values.

6. The device as set forth in claim 4, wherein the flow sensor comprises an analog/digital-converter which is integrated on a chip and digitally transfers the data captured.

7. The device as set forth in claim 4, wherein the data transfer means comprises cables which lead from the flow sensor in the catheter head along the liquid supply to a processing unit.

8. The device as set forth in the preceding claim, wherein said processing unit is the regulating means on the pump.

9. The device as set forth in claim 7, wherein line junctions at components of the liquid supply to be connected to each other are realized by contacts.

10. The device as set forth in the preceding claim, wherein said contacts are touching contacts.

11. The device as set forth in claim 4, wherein the data transfer means comprises cables which lead separately from the liquid supply to a processing unit.

12. The device as set forth in the preceding claim, wherein said processing unit is the regulating means on the pump.

13. The device as set forth in claim 11, wherein it is possible to connect the cables to the catheter head and fix them there by means of a plug-on adapter.

14. The device as set forth in claim 4, wherein the data transfer means comprises a transmitter which wirelessly transfers the data captured by the flow sensor to a receiver on a processing unit.

15. The device as set forth in the preceding claim, wherein said processing unit is the regulating means on the pump.

16. The device as set forth in claim 14, wherein it is possible to connect the transmitter to the catheter head and fix it there by means of a plug-on adapter.

17. A device for supplying liquid into a person's body, comprising a catheter head, a liquid supply to said catheter head and a flow sensor associated with the catheter head.

18. The device according to claim 17, wherein the flow sensor comprises an analog/digital-converter which is integrated on a chip and digitally transfers flow data.

19. The device according to claim 17, further comprising a processing unit and a pump, operably coupled to the flow sensor in a feedback loop.

20. A device for supplying liquid into a person's body, comprising a catheter head, a pump, a conduit through which the liquid may flow coupled to said catheter head and pump, a flow sensor carried by the catheter head, and a processing unit, said flow sensor, processing unit and pump operably coupled in a feedback loop.
